(19) United States
(12) Patent Application Publication
(10) Pub. No.: US 2005/0214747 A1
(43) Pub. Date: Sep. 29, 2005

(54) Compositions and methods for analysis of target analytes

(76) Inventors: Robert Danielzadeh, San Jose, CA (US); Keith R. Olson, Oakland, CA (US)

Correspondence Address: Aldo J. Test;Dorsey & Whitney LLP, Intellectual Property Department, 850 Hansen Way, Suite 200, Palo Alto, CA, 94304-1017, US

(21) Appl. No.: 10/969,170

(22) Filed: Oct. 17, 2004

Related U.S. Application Data
(60) Provisional application No. 60/537,261, filed on Jan. 16, 2004.

(51) Int. Cl. C12Q 1/70; C12Q 1/68; G01N 33/53

(52) U.S. Cl. 435/5; 435/6; 435/7.1

(57) ABSTRACT

Compositions and methods are provided for analyzing a sample for the presence or absence of one or more target analytes.

### 1. PRIORITY AND RELATED APPLICATIONS

This application claims the benefit for the filing dates of U.S. Provisional Patent Application Ser. No. 60/504,563, filed Sep. 17, 2003, pending, and U.S. Provisional Patent Application Ser. No. 60/537,261, filed Jan. 16, 2004, pending. All priority and related applications are hereby incorporated by reference in their entirety.

### 2. FIELD

The present disclosure relates to compositions and methods for detection of one or more target analytes in samples.

### 3. BACKGROUND

Analytical methods are important for research and clinical testing. For example, the analysis of molecules with biological activities and/or functions have provided methods and compositions for the diagnosis and treatments of disease states. As a result of the increasing amount of information becoming available about the structure and function biological molecules, including the entire sequence of the human genome, methods of analyzing such molecules will play a more prominent role in research, diagnosis, treatment, and prevention. Methods that are rapid, convenient and sensitive and can be used to analyze multiple targets (e.g., cells, secreted molecule, and intracellular targets) simultaneously will have broad application.

There is accordingly, a need in the art for methods and compositions than can be adapted for detection, quantitation, and/or characterization of one or more extracellular and/or intracellular analytes.

### 4. SUMMARY

In one aspect, the present disclosure provides a method of detecting a target analyte. The method comprises labeling, in a vessel, a first target analytes that is cell associated and a second target analyte that is not cell associated with moieties capable of producing detectable signals and detecting the signals produced by the labeled target analytes.

In one embodiment, the first target analyte is a precursor of the second analyte. In one embodiment, the first and second analytes independently comprise a peptide, a nucleic acid, a carbohydrate, a lipid, or combinations thereof. In one embodiment, the first and second target analytes are virus peptides, nucleic acids, or combinations thereof. In one embodiment, the moieties capable of producing a detectable signals are fluorescent moieties. In one embodiment, one of the target analytes can be labeled by binding to a microparticle. In one embodiment, the signals are detected by a microcapillary cytometer.

In another aspect, the present disclosure provides a method of detecting a target analyte. The method comprises inhibiting binding partner—target analyte binding with a microparticle comprising a competitive inhibitor of the target analyte, and measuring the binding partner bound to the competitive inhibitor as the microparticle is drawn through a microcapillary cytometer that is optically linked to a fluorescence system.

In one embodiment, the binding partner is an antibody. In one embodiment, the binding partner comprises a fluorescent moiety. In one embodiment, the binding partner bound to the competitive inhibitor is labeled with a fluorescent moiety. In embodiment, the binding partner is labeled by binding to an anti-binding partner comprising a fluorescent moiety. In some embodiments, the method further comprises quantitating the target analyte.

In another aspect is provided a method of detecting a target analyte. The method comprises, reacting an antibody with a target analyte and a competitive inhibitor thereof under competitive binding conditions, and measuring the antibody bound to said competitive inhibitor as it is drawn through a microcapillary cytometer that is optically linked to a detection system.

### 5. BRIEF DESCRIPTION OF THE DRAWINGS

The skilled artisan will appreciate that the drawings, described below, are for illustration only and are not intended to limit the scope of the present disclosure.

FIG. 1 is a cartoon depicting an embodiment of a competitive inhibition assay. In the depicted embodiment, primary antibody B 130 (first binding partner, anti-target analyte) is added to a mixture containing target analyte 180 (X_{ca}) and inhibitor 110 thereof (X) labeled with bead or microparticle 120 that competes with target analyte 180 binding to primary antibody 130. Primary antibody 130 that does not bind X-bead 160 (A) is removed. Secondary antibody 140 that binds to primary antibody 130 and has moiety 150 (PE) capable of producing a detectable signal is added to form complex 100 comprising X-bead 160, primary antibody 130 and PE labeled secondary antibody 170. Secondary antibody 170 that does not bind to primary antibody 130 is removed and the complex is detected by a microflow cytometer.

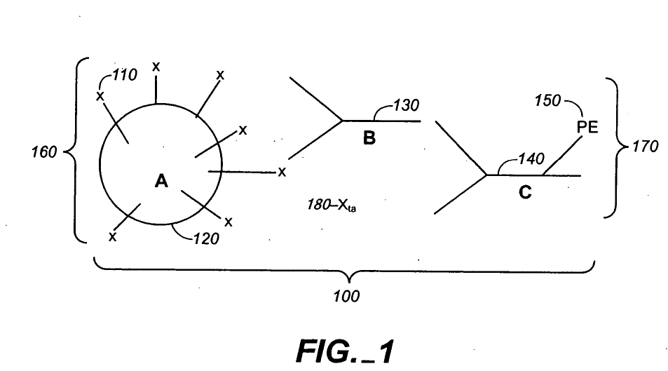

FIG. 2 shows the results of the isotype negative control antibody of Example 1, which does not bind to insulin, detected by a microcapillary cytometry (Guava PCA, Guava Technologies, Hayward, Calif.).

FIG. 3 shows the results of the analysis of the inhibitor control of Example 1 as detected by microcapillary cytometry (Guava PCA, Guava Technologies, Hayward, Calif.).

FIG. 4 shows the results of the analysis of the complex of Example 1 consisting of inhibitor/primary antibody/fluorescence labeled secondary antibody detected by a microcapillary cytometry (Guava PCA, Guava Technologies, Hayward, Calif.).

FIG. 5 shows the inhibition of primary antibody binding to insulin as described in Example 1. The inhibition is in comparison to FIG. 4.

FIG. 6 is a graph of the competitive binding between insulin and insulin inhibitor for anti-insulin antibody. As the concentration of insulin increases the amount of antibody available for binding to inhibitor decreases resulting in a decrease in MFI (see Example 1).

FIG. 7 is an example of “doublet” phenomenon resulting from non-specific binding of microparticles to each other. Doublet phenomenon not observed or substantially decreased by the methods disclosed herein.

FIG. 8 shows the analysis of beads alone, cells alone, and cells+beads by microcapillary cytometry (Guava PCA, Guava Technologies, Hayward, Calif.). Panel A: analysis by fluorescence detection. Panel B: analysis by light scatter.

FIG. 9 shows the analysis of beads of various fluorescence intensities and cells by microcapillary cytometry (Guava PCA, Guava Technologies, Hayward, Calif.). Panel A: analysis by fluorescence detection. Panel B: analysis by light scatter.

FIG. 10 shows the simultaneous analysis of live cells, dead cells, and beads (see Example 5).

FIG. 11 shows a graph of mean intensity (PM 1) vs. monoclonal antibody concentration (see Example 7).

### 5. DETAILED DESCRIPTION

The disclosure provides compositions and methods for detecting and/or quantitating one or more target analytes.

In some embodiments the disclosure provides compositions and methods for detecting one or more target analyte(s) that is cell-associated (ca-target analyte) and one or more target analyte that is not cell associated (na-target analyte). In some embodiments, the ca- and na-target analytes can be labeled with a moiety capable of producing a detectable signal. In some embodiments, the ca- and a na-target analyte can be directly or indirectly labeled in a single reaction vessel with moieties capable of producing detectable signals. In some embodiments, one or more detectable moieties can be a microparticle.

In some embodiments, a target analyte can be detected under competitive binding conditions, in which the target analyte and an inhibitor thereof compete for binding to a binding partner of the target analyte. In some embodiments, competitive binding conditions can be established by determining the range of concentration of the binding partner that may be insufficient to bind all of the inhibitor and target analyte present but provides a detectable signal above background. Therefore, in various exemplary embodiments, the amount of binding partner can be sufficient to bind from about 10% to ≦100% of the inhibitor, from about 10% to less than about 75% of the inhibitor, from about 10% to less than about 50% of the inhibitor, or about 10% to less than about 25% of the inhibitor. Detecting the binding partner that binds to the target analyte and/or inhibitor can be an indicator of the presence or absence of the target analyte. In some embodiments, measuring the binding partner bound to the inhibitor can be used to quantitate the target analyte. In some embodiments, the binding partner can be directly or indirectly labeled with a moiety suitable for producing a detectable signal. In some embodiments, the inhibitor can be labeled with a microparticle.

In some embodiments, competitive binding conditions can be used to detect or characterize a binding partner. Therefore, in some embodiments, a ligand, a first binding partner of the ligand, and a sample, which may contain a second binding partner, react under competitive binding conditions. The inhibition of binding of the first binding partner and ligand can be indicative of the presence and/or the affinity of a second binding partner in the sample. In some embodiments, the first binding partner can be directly or indirectly labeled with a moiety suitable for producing a detectable signal. In some embodiments, the ligand can be labeled.

The skilled artisan will appreciate that the product of the methods disclosed herein (e.g., target analyte/binding partner, inhibitor/binding partner, and ligand/binding partner complexes) can be detected and/or quantitated by various methods as known in the art. However, in some embodiments, the complexes can be detected and/or quantitated by a microcapillary cytometer that is optically coupled to a detection system. In various exemplary embodiments, the complexes can be detected by forward light scatter and/or a signal produced by one or more detectable moieties.

By “target analyte”, “analyte” and grammatical equivalents herein are meant a substance capable of being analyzed (e.g., detected, quantitated, and/or characterized) by the disclosed methods. In some embodiments “capable of being detected” refers to a target analyte having at least one property, for example, size, shape, dimension, binding affinity, or a detectable moiety that renders the target analyte suitable for analysis by the disclosed methods. In some embodiments, a target analyte can intrinsically comprise a property that can be analyzed by the disclosed methods. In some embodiments, a target analyte can be modified to comprise a property that can be analyzed by the disclosed methods. Thus, in some embodiments a target analyte can bind to one or more other substances directly or indirectly to form a complex having at least one property suitable for analysis. Thus, in some embodiments a target analyte can be bound to any number of substances selected at the discretion of the practitioner. Selecting the number and types of target analytes is within the abilities of the skilled artisan.

In some embodiments, a target analyte can be cell-associated. By “cell-associated” herein is meant bound, connected, contained by a cell. Therefore, in various exemplary embodiments, cell-associated includes but is not limited to target analytes bound to a cell (e.g., bound to cell receptor) and/or being associated with a cellular structure and/or being internal to the most exterior membrane of a cell (e.g. intracellular). For example, a target analyte can be a nuclear, cytoplasmic, or mitochondrial constituent. In some embodiments, a cell-associated target analyte may be a component of a cell wall, a cell membrane, or a periplasmic region. In some embodiments, a target analyte is not cell-associated (na-target analyte). Therefore, a target analyte may not be bound, connected, or contained by a cell (extracellular). The skilled artisan will appreciate that in some embodiments, a target analyte can be cell-associated and be released or secreted by a cell and accordingly may become extracellular. Therefore, in some embodiments a cell-associated target analyte can be a precursor of a target analyte that is not cell-associated.

In various exemplary embodiments a target analyte includes but is not limited to a molecule (e.g., polynucleotides (e.g., nucleic acid sequence, plasmid, chromosome, DNA, RNA, cDNA etc.), polypeptides (e.g., antibodies, receptors, hormones, cytokines, CD antigens, MHC molecules, enzymes (e.g. proteases, serine proteases, metalloproteases as the like), an organic compound (e.g., steroids, sterols, carbohydrates, lipids), an inorganic compound), a carbohydrate, a lipid, microparticle (e.g., a microbead, a lipid vesicle (e.g., liposome or exosome), a cell (e.g., eukaryotic and prokaryotic cells), a cell fragment (e.g., a membrane fragment, sacculi, a nucleus, a mitochondria, a Golgi, a vesicle, endoplasmic reticulum and other organelles), a corpuscle (e.g., a mammalian erythrocyte), platelet, a virus (e.g., Adenoviruses, Herpesviruses, Papillomaviruses, Polyomaviruses, Poxviruses, Parvoviruses, Hepadnaviruses, Retroviruses, Reoviruses, Arenaviruses, Bornaviruses, Bunyaviruses, Filoviruses, Orthomyxoviruses, Paramyxoviruses, Rhabdoviruses, Filoviruses, Arteriviruses, Astroviruses, Caliciviruses, Coronaviruses, Flaviviruses, “Hepatitis E-like viruses”, Picornaviruses, Togaviruses, Bornaviruses, Prions etc.), and combinations thereof.

In some embodiments a product formed by the disclosed methods may have a diameter of about 150 nm to about 40 μm. However, the skilled artisan is aware that the size or volume of the product and its suitability for use in the disclosed methods can be at least determined in part by the method selected for detection, as described below. Therefore, products having smaller and larger diameters also are contemplated by the present disclosure. However, the skilled artisan appreciates that the size of the product can result in a signal that can be off scale or a signal beneath the detection threshold. Determining the optimum size of the product for detection is within the abilities of the skilled artisan. Although in some embodiments the product volume may be calculated from the radius, in some embodiments a product of the disclosed methods may not be spherical. Therefore, also contemplated are products that may be irregularly shaped, cubical, oval, elongated, and the like.

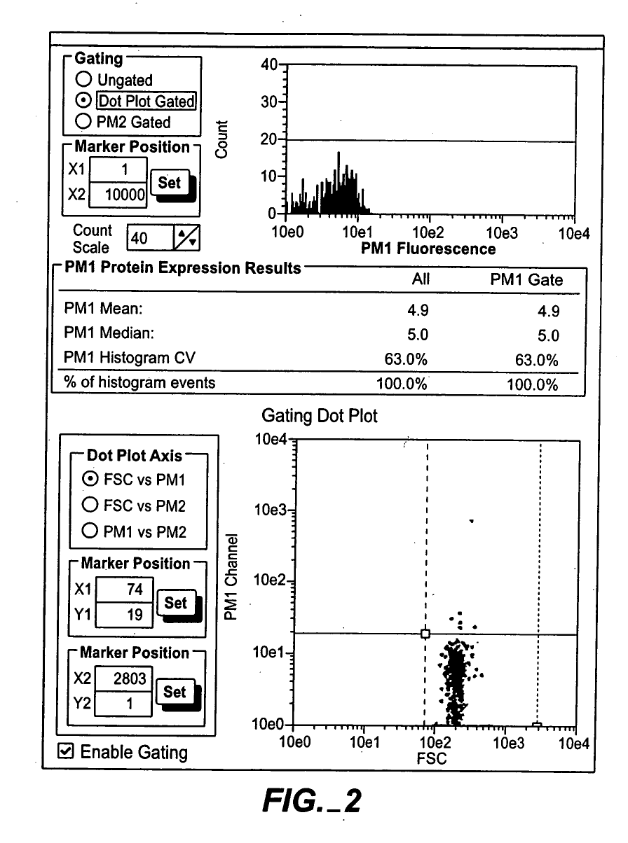

By “polynucleotide”, “nucleic acid sequence” and grammatical equivalents herein are meant a nucleobase sequence, including by not limited to, DNA, cDNA, RNA (e.g., mRNA, rRNA, vRNA, iRNA), a product of an amplification process (Polymerase Chain Reaction (PCR), Ligase Chain Reaction (LCR), Strand Displacement Amplification (SDA; Walker et al., 1989, Proc. Natl. Acad. Sci. USA 89:392-396; Walker et al., 1992, Nucl. Acids Res. 20(7):1691-1696; Nadeau et al., 1999, Anal. Biochem. 276(2):177-187; U.S. Pat. Nos. 5,270,184, 5,422,252, 5,455,166, 5,470,723), Transcription-Mediated Amplification (TMA), Q-beta replicase amplification (Q-beta), Rolling Circle Amplification (RCA; Lizardi, 1998, Nat. Genetics 19(3):225-232 and U.S. Pat. No. 5,854,033), Asymmetric PCR (Gyllensten et al., 1988, Proc. Natl. Acad. Sci. USA 85:7652-7656) or Asynchronous PCR (WO 01/94638)) or a product of a synthetic process (see U.S. Pat. Nos. 5,258,454, 5,373,053). As outlined herein, the polynucleotide may be of any length suitable for analysis by the disclosed methods, with the understanding that longer sequences are more specific in their hybridization to a complementary sequence. “Nucleobase” refers to those naturally occurring and those synthetic nitrogenous, aromatic moieties commonly found in the nucleic acid arts. Examples of nucleobases include purines and pyrimidines, genetically encoded nucleobases, analogs of genetically encoded nucleobases, and purely synthetic nucleobases. Specific examples of genetically encoded bases include adenine, cytosine, guanine, thymine, and uracil. Specific examples of analogs of genetically encoded bases and synthetic bases include 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). 5-propynyl-uracil, 2-thio-5-propynyl-uracil. Other non-limiting examples of suitable nucleobases include those nucleobases illustrated in FIGS. 2(A) and 2(B) of U.S. Pat. No. 6,357,163, incorporated herein by reference in its entirety.

Nucleobases can be linked to other moieties to form nucleosides, nucleotides, and nucleoside/tide analogs. As used herein, “nucleoside” refers to a nucleobase linked to a pentose sugar. Pentose sugars include ribose, 2′-deoxyribose, 3′-deoxyribose, and 2′,3′-dideoxyribose. “Nucleotide” refers to a compound comprising a nucleobase, a pentose sugar and a phosphate. Thus, as used herein a nucleotide refers to a phosphate ester of a nucleoside, e.g., a triphosphate. Nucleic acid analogs, including nucleoside and nucleotide analogs, are described below.

By “nucleic acid” or “oligonucleotide” and their grammatical equivalents herein are meant at least two nucleotides covalently linked together. A nucleic acid of the present disclosure will generally contain phosphodiester bonds, although in some cases, as outlined below, nucleic acid analogs are included that may have alternate backbones, comprising, for example, phosphoramide (Beaucage et al., 1993, Tetrahedron 49(10): 1925 and references therein; Letsinger, 1970, J. Org. Chem. 35:3800; Sprinzl et al., 1977, Eur. J. Biochem. 81:579; Letsinger et al., 1986, Nucl. Acids Res. 14:3487; Sawai et al., 1984, Chem. Lett. 805, Letsinger et al., 1988, J. Am. Chem. Soc. 110:4470; and Pauwels et al., 1986, Chemica Scripta 26:141), phosphorothioate (Mag et al., 1991, Nucleic Acids Res. 19:1437; and U.S. Pat. No. 5,644,048), phosphorodithioate (Briu et al., 1989, J. Am. Chem. Soc. 111:2321) O-methylphophoroamidite linkages (Eckstein, Oligonucleotides and Analogues: A Practical Approach, Oxford University Press), and peptide nucleic acid backbones and linkages (Egholm, 1992, J. Am. Chem. Soc. 114:1895; Meier et al., 1992, Chem. Int. Ed. Engl. 31:1008; Nielsen, 1993, Nature 365:566; Carlsson et al., 1996, Nature 380:207, all of which are incorporated by reference). Other analog nucleic acids include those with bicyclic structures including locked nucleic acids (LNAs), Koshkin et al., 1998, J. Am. Chem. Soc. 120:13252-3; positive backbones (Denpcy et al., 1995, Proc. Natl. Acad. Sci. USA 92:6097; non-ionic backbones (U.S. Pat. Nos. 4,469,863, 5,216,141, 5386023, 5602240, 5637684, Kiedrowshi et al., 1991, Angew. Chem. Intl. Ed. English 30:423; Letsinger et al., 1988, J. Am. Chem. Soc. 110:4470; Letsinger et al., 1994, Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al., 1994, Bioorganic & Medicinal Chem. Lett. 4:395; Jeffs et al., 1994, J. Biomolecular NMR 34:17) and non-ribose backbones, including those described in U.S. Pat. Nos. 5,034,506, 5,235,033 and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (Jenkins et al., 1995, Chem. Soc. Rev. pp. 169-176). Several nucleic acid analogs are described in Rawls, C & E News Jun. 2, 1997, page 35. All of these references are hereby expressly incorporated by reference. The modifications of the ribose-phosphate backbone may be done to facilitate the addition of various moieties as known in the art, or to increase the stability and half-life of such molecules in physiological environments.

As will be appreciated by those in the art, all of these nucleic acid analogs may find use in the present invention. In addition, mixtures of naturally occurring nucleic acids and analogs can be made. Alternatively, mixtures of different nucleic acid analogs, and mixtures of naturally occurring nucleic acids and analogs may be made.

In some embodiments nucleic acid analogs are peptide nucleic acids (PNA), and peptide nucleic acid analogs. “Peptide Nucleic Acid” or “PNA” refers to nucleic acid analogs in which the nucleobases are attached to a polyamide backbone through a suitable linker (e.g., methylene carbonyl, aza nitrogen) such as described in any one or more of U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470, 6,451,968, 6,441,130, 6,414,112, 6,403,763, all of which are incorporated herein by reference. PNA backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tₘ) for mismatched versus perfectly matched base pairs. DNA and RNA typically exhibit about a 2-4° C. drop in Tₘ for an internal mismatch. With the non-ionic PNA backbone, the drop is closer to about 7-9° C. This allows for better detection of mismatches. Similarly, due to their non-ionic nature, hybridization of the bases attached to these backbones can be relatively insensitive to salt concentration.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc. Some embodiments utilize isocytosine and isoguanine in nucleic acids designed to be complementary to other nucleic acids as this reduces non-specific hybridization, as generally described in U.S. Pat. No. 5,681,702. Some embodiments utilize diaminopurines (see e.g., Haaima et al., 1997, Nucleic Acids Res., 25: 46394643; and Lohse et al., 1999, Proc. Natl. Acad. Sci. USA 96: 11804-11808).

The ability to determine hybridization conditions between nucleic acid or nucleobases sequences is known in the art and is described, for example, in Baldino et al. Methods Enzymology 168:761-777; Bolton et al., 1962, Proc. Natl. Acad. Sci. USA 48:1390; Bresslauer et al., 1986, Proc. Natl. Acad. Sci. USA 83:8893-8897; Freier et al., 1986, Proc. Natl. Acad. Sci. USA 83:9373-9377; Kierzek et al., Biochemistry 25:7840-7846; Rychlik et al., 1990, Nucleic Acids Res. 18:6409-6412 (erratum, 1991, Nucleic Acids Res. 19:698); Rychlik. J. NIH Res. 6:78; Sambrook et al. Molecular Cloning: A Laboratory Manual 9.50-9.51, 11.46-11.50 (2d. ed., Cold Spring Harbor Laboratory Press); Sambrook et al., Molecular Cloning: A Laboratory Manual 10.1-10.10 (3d. ed. Cold Spring Harbor Laboratory Press); Suggs et al., 1981, In Developmental Biology Using Purified Genes (Brown et al., eds.), pp. 683-693, Academic Press; Wetmur, 1991, Crit. Rev. Biochem. Mol. Biol. 26:227-259.

By “polypeptide” and grammatical equivalents herein are meant at least two covalently attached amino acids, which includes proteins, oligopeptides and peptides. The polypeptide may be made up of naturally occurring amino acids and peptide bonds, or synthetic peptidomimetic structures, i.e. “analogs”, such as peptoids (see Simon et al., 1992, Proc. Natl. Acad. Sci. USA 89(20):9367). Thus “amino acid” or “peptide residue” as used herein means both naturally occurring and synthetic amino acids. For example, homophenylalanine, citrulline and noreleucine are considered amino acids for the purposes of the invention. “Amino acid” also includes imino acid residues such as proline and hydroxyproline. The side chain may be in either the (R) or the (S) configuration. In the preferred embodiment, the amino acids are in the (S) or (L) configuration. If non-naturally occurring side chains are used, non-amino acid substituents may be used, for example to prevent or retard in vivo degradation. In some embodiments a polypeptide contains non-polypeptide constituents, including but not limited, to N-linked carbohydrate, O-linked carbohydrate, fatty acids.

Various exemplary embodiments of polypeptides include but are not limited to a hormone (e.g., insulin, growth hormone (GH), erythropoietin (EPO), thyroid-stimulating hormone (TSH), follicle-stimulating hormone (FSH), luteinizing hormone (LH), prolactin (PRL), adrenocorticotropic hormone (ACTH), antidiuretic hormone (ADH), oxytocin, thyrotropin-releasing hormone (TRH), gonadotropin-releasing hormone (GnRH), growth hormone-releasing hormone (GHRH), corticotropin-releasing hormone (CRH), somatostatin, calcitonin, parathyroid hormone (PTH), gastrin peptides, secretin peptide, cholecystokinin (CCK), neuropeptide Y, ghrelin, PYY3-36 peptide, insulin-like growth factors (IGFs), angiotensinogen, thrombopoietin, leptin), cluster designation antigens (e.g., CD1, CD2, CD3, CD4, CD5, CD6, CD7, CD8, CD11a, CD11b, CD11c, CD13, CD14, CD15, CD19, CD20, CD21, CD22, CD25, CD33, CD34, CD37, CD38, CD41, CD42b, CD45, CD68, CD71, CD79a, CD80, CD138), chemokines/cytokines (e.g., interleukins (e.g, IL-1, -2, -3, 4, -5, -6, -7, -8, -9, -10, -11, -12, -13, -14, -15); BDNF, CREB pS133, CREB, DR-5, EGF, Eotaxin, Fatty Acid Binding Protein, FGF-basic, G-CSF, GCP-2, GM-CSF, GRO-KC, HGF, ICAM-1, IFN-α, IFN-γ, IP-10, JE/MCP-1, KC, KC/GROa, LIF, lymphotacin, M-CSF, MCP-1, MCP-1 (MCAF), MCP-3, MCP-5, MDC, MIG, MIP-1, MIP-1 β, MIP-1 γ, MIP-2, MIP-3 β, OSM, PDGF-BB, RANTES, Rb (pT821), Rb (total), Rb pSpT249/252, Tau (pS214), Tau (pS396), Tau (total), TNF-α TNF-β, TNF-RI, TNF-RII, VCAM-1, VEGF), major histocompatibility antigens (e.g., MHC-I, MHC-II, MHC-III, HLA (human: e.g., B, C, A, DQ, DA, DR, DP), H-2 (mouse: e.g., Ia, lb, K, D, L), RTI (rat: e.g., A, H, C/E)), receptors (e.g., T-cell receptor, insulin receptor), cell surface antigens (e.g., Gr-1), antibodies (e.g., IgG, IgM, IgA, IgD, IgE, monoclonal antibody (MAb), polyclonal antibody, Fab, Fab′, F(ab′)₂, Fᵥ, single-chain antibody, chimeric antibody, humanized antibody), viral proteins (e.g., HIV (e.g., gp120, gp41, p24), HBV (e.g., hepatitis B surface antigen), SARS (e.g., S protein)), enzymes (e.g., alkaline phosphates, caspases, tyrosine kinases, serine kinases, proteases, glycosylases, phosphatases, polymerases, transcriptases) and transcription factors.

By “carbohydrate” and grammatical equivalents herein are meant compounds of carbon, hydrogen, and oxygen containing a saccharose grouping or its first reaction product, and in which the ratio of hydrogen to oxygen is the same as water, and derivates thereof. (“Encyclopedia of Chemistry, 4^{th} Ed. (ISBN 0-442-22572-2)) Thus, carbohydrate includes but is not limited to monosaccharides, oligosaccharides and polysaccharides compounds derived from monosaccharides by reduction of the carbonyl group, by oxidation of one or more terminal groups to carboxylic acids, or by replacement of one or more hydroxy group(s) by a hydrogen atom, an amino group, a thiol group or other heteroatomic groups. Thus, various exemplary embodiments of carbohydrate include but are not limited to aldoses, ketoses, hemiacetals, hemiketals, furanoses, pyranoses, ketoaldoses (aldoketoses, aldosuloses), deoxy sugars, amino sugars, alditols, aldonic acids, ketoaldonic acids, uronic acids, aldaric acids, glycosides, and linear and branched homo- and hetero-polymers thereof.

By “cell” and grammatical equivalents herein are meant the smallest unit of living structure, composed of a membrane-enclosed mass of protoplasm and containing a nucleus or nucleoid, and fragments and subcomponents thereof. In some embodiments a cell can be capable of carrying out at least one biological function or biochemical reaction including but not limited to a catabolic or anabolic pathway or reaction, cell division (e.g., mitosis, meiosis, binary fission), apoptosis, chemotaxis, immune recognition, etc. In some embodiments a cell can be non-viable or incapable of carrying out such functions or reactions. In some embodiments a cell can be treated with a composition, including a pharmaceutical composition, a toxin, a metabolite, a hormone, an immune modulator (cytokine, interleukin, chemokine etc), a nucleic acid, a polypeptide, a virus and the like.

By “eukaryotic cell” and grammatical equivalents herein are meant a cell containing a membrane-bound nucleus with chromosomes of DNA, RNA, and proteins, and subcellular structures, such as mitochondria or plastids. Examples of eukaryotic cells include but are not limited to the cells of protists, protozoa, fungi, plants, and animals. Thus, in various exemplary embodiments a eukaryotic cell can be obtained from an in vitro culture, or a living or deceased organism, including but not limited to primates, rodents, lagomorphs, canines, felines, fish, reptiles, nematodes, cestodes, trematodes, helminths, transgenic animals, knock-out animals, cloned animals, insects and microorganisms (e.g., flagellates, ciliates, amoebas, yeast, fungi), including developmentally immature or dormant forms thereof (e.g., a neonate, a fetus, an embryo, a spore, forms found in intermediate hosts and the like). In a preferred embodiment, a eukaryotic cell can be a human cell, including by not limited to, a lymphocyte, including T-cells and B-cells, macrophages, neutrophils, basophils, eosinophils, gametes, and cells obtained from a biopsy or tissue sample. In some embodiments a eukaryotic cell can be a non-nucleated cell such as a red blood cells or corpuscles, which in humans lose their nucleus as part of their maturation process. In another preferred embodiment, a eukaryotic cell can be a cell of a human neonate. In another preferred embodiment, a eukaryotic cell can be infected, productively or non-productively, with a microorganism, including but not limited to, a virus (e.g., human immunodeficiency virus (HIV), human T-cell leukemia viruses (HTLVs), herpes simplex viruses (HSV-I, -II), cytomegalovirus (CMV), dengue virus (DV)), a bacterium (e.g., Mycobacterium, Salmonella, Rickettsia) or a protozoa (e.g., Plasmodium, Leishmania, Trypanosoma). In some embodiments a cell can be a malignant cell, including but not limited to, a leukemic cell (e.g., acute lymphocytic leukemia (ALL), acute myelogenous leukemia (AML), chronic lymphocytic leukemia (CLL), chronic myelogenous leukemia (CML)), a melanoma, hepatoma, glioma, neuroblastoma, myeloma, and colon, prostate, breast, and cervical cancer cell. In some embodiments, a cell can be a hybrid cell (e.g., a hybridoma).

By “prokaryotic cell” and grammatical equivalents herein are meant a cell which lacks, for example, a nuclear membrane, paired organized chromosomes, a mitotic mechanism for cell division, and mitochondria. Examples of prokaryotic cells include but are not limited to cyanobacteria (e.g., blue-green bacteria), archaebacteria (e.g., methanogens, halophiles, thermoacidophiles), and eubacteria (e.g., heterotrophs, autotrophs, chemotrophs). Thus, in some embodiments the prokaryotic cell can be Gram positive, Gram negative, aerobic, anaerobic, or facultative anaerobic. Accordingly, prokaryotic cells include but are not limited to Acinetobacter, Aeromonas, Alcaligenes, Bacillus, Bordetella, Borriela, Branhamella, Campylobacter, Chlamydia, Clostridium, Corynebacterium, Escherichia, Enterobacter, Hafnia, Haemophilus, Helicobacter, Klebsiella, Lactobacillus, Listeria, Micrococcus, Morganella, Mycobacterium, Neisseria, Propionbacter, Providencia, Proteus, Pyrococcus, Salmonella, Serratia, Shewanella, Shigella, Staphylococcus, Streptococcus, Thermophilus, Vibrio, Yersinia. In some embodiments, a prokaryotic cell can be infected with a microorganism, such as, as virus (e.g., T4, T7, M13, and other phage).

In some embodiments, a target analyte can be an organic compound, including but not limited to a member of a chemical library, a pharmaceutical (e.g., an antibiotic (e.g., erythromycin, penicillin, methicillin, gentamicin), an antiviral (e.g., amprenavir, indinavir, saquinavir, saquinavir, lopinavir, ritonavir, fosamprenavir, ritonavir, atazanavir, nelfmavir, tipranavir), a chemotherapeutic (e.g., doxorubicin, denileukin diftitox, fulvestrant, gemcitabine, taxotere)), a controlled substance (e.g., cocaine, heroine, THC, LSD), a barbiturate (e.g., amobarbital, aprobarbital, butabarbital, butalbital, hexobarbital, mephobarbital, morphine, pentobarbital, phenobarbital, secobarbital, sodium pentothal, thiopental), an amphetamine, a steroid (e.g., oxymethalone, oxandralone, methandrostenalone, stanozolol, nandrolone, depo-testosterone, androgens, estrogens).

In some embodiments, a target analyte can be analyzed under competitive binding conditions. By “competitive binding conditions” and grammatical equivalents herein are meant reaction conditions in which a target analyte and another compound (“inhibitor”) compete for binding to a binding partner. In some embodiments, the target analyte and inhibitor compete for binding to the same or substantially same site of the binding partner. In some embodiments, the target analyte and inhibitor bind to different sites of the binding partner, however, the binding of the target analyte or the inhibitor substantially decreases the affinity of the binding partner for the other compound. In some embodiments, the inhibition can be mixed (see, e.g., Nelson and Cox, Lehninger Principles of Biochemistry 265-269 (3d ed. Worth Publishers, 2000)).

Therefore, in some embodiments, the structure of an inhibitor can be substantially equivalent to a target analyte or substantially equivalent to the portion or region of a target analyte that binds to the binding partner. In some embodiments, the chemical structure of an inhibitor can be substantially different than the target analyte but mimic the three-dimensional structure of a target analyte. Therefore, in some embodiments, an inhibitor can be a mimetope. However, the skilled artisan will appreciate that in some embodiments the chemical and three-dimensional structures of a target analyte and an inhibitor thereof can be at least substantially unique.

In some embodiments, an inhibitor comprises a microparticle. By “microparticle”, “microsphere”, “microbead”, “bead” and grammatical equivalents herein are meant a small discrete synthetic particle. As known in the art, the composition of beads will vary depending on the type of assay in which they are used and, therefore, the composition can be selected at the discretion of the practitioner. Suitable bead compositions include those used in peptide, nucleic acid and organic synthesis, including, but not limited to, plastics, ceramics, glass, polystyrene, methylstyrene, acrylic polymers, paramagnetic materials (U.S. Pat. Nos. 4,358,388; 4,654,267; 4,774,265; 5,320,944; 5,356,713), thoria sol, carbon graphite, titanium dioxide, latex or cross-linked dextrans such as Sepharose, agarose, cellulose, carboxymethyl cellulose, hydroxyethyl cellulose, proteinaceous polymer, nylon, globulin, DNA, cross-linked micelles and Teflon may all be used. “Microsphere Detection Guide” from Bangs Laboratories, Fishers, Ind. is a helpful guide. Beads are also commercially available from, for example, Bio-Rad Laboratories (Richmond, Calif.), LKB (Sweden), Pharmacia (Piscataway, N.J.), IBF (France), Dynal Inc. (Great Neck, N.Y.). In some embodiments, beads may contain a cross-linking agent, such as, but not limited to divinyl benzene, ethylene glycol dimethacrylate, trimethylol propane trimethacrylate, N,N'methylene-bis-acrylamide, adipic acid, sebacic acid, succinic acid, citric acid, 1,2,3,4-butanetetracarboxylic acid, or 1,10 decanedicarboxylic acid or other functionally equivalent agents known in the art. In various exemplary embodiments, beads can be spherical, non-spherical, egg-shaped, irregularly shaped, and the like. The average diameter of a microparticle can be selected at the discretion of the practitioner. However, generally the average diameter of microparticle can range from nanometers (e.g. about 100 nm) to millimeters (e.g. about 1 mm) with beads from about 0.2 μm to about 200 μm being preferred, and from about 0.5 to about 10 μm being particularly preferred, although in some embodiments smaller or larger beads may be used, as described below.

In some embodiments a microparticle can be porous, thus increasing the surface area of the available for attachment to another molecule, moiety, or compound (e.g., an inhibitor) as described below. Thus, microparticles may have additional surface functional groups to facilitate attachment and/or bonding. These groups may include carboxylates, esters, alcohols, carbamides, aldehydes, amines, sulfinur oxides, nitrogen oxides, or halides. Methods of attaching another molecule or moiety to a bead are known in the art (see, e.g., U.S. Pat. Nos. 6,268,222, 6,649,414). In alternative emodiments, a microparticle can further comprise a label, e.g., a fluorescent label or may not further comprise a label.

In some embodiments, a microparticle can be a lipid vesicle. By “lipid vesicle”, “liposome” and grammatical equivalents herein are meant a continuous and/or non-continuous lipid surface, either unilamellar or multilamellar, enclosing a three-dimensional space. In some embodiments an inhibitor can comprise a lipid vesicle. Included within the meaning of “lipid vesicle” are liposomes and naturally occurring lipid vesicles, such endocytic or exocytic vesicles and exosomes from a cell, including but not limited to a dendritic cell (see, e.g., Chaput et al., 2003, Cancer Immunol Immunother. 53(3):234-9; Estevez et al., 2003, J Biol. Chem. 278(37):34943-51; Evguenieva-Hackenburg et al., 2003, EMBO Rep. 4(9):889-93; Gould et al., 2003, Proc Natl Acad Sci USA 100(19):10592-7; Haile et al., 2003, RNA 9(12):1491-501; Hawari et al., 2004, Proc Natl Acad Sci USA 101(5): 1297-302; Mitchell et al., 2003, Mol Cell. 11(5):1405-13; Mitchell et al., 2003, Mol Cell Biol. 23(19):6982-92; Nguyen et al., 2003, J. Biol. Chem. 278(52):52347-54; Phillips et al., 2003, RNA 9(9):1098-107; Raijmakers et al., 2003, J Biol. Chem. 278(33):30698-704; Savina et al., 2003, J Biol. Chem. 278(22):20083-90); Tran et al., 2004, Mol Cell. 13(1):101-11; Yehudai-Resheff et al., 2003, Plant Cell. 15(9):2003-19). Thus, in various exemplary embodiments, an inhibitor can be incorporated by the practitioner into a lipid vesicle or can be a naturally-occurring component of a lipid vesicle.

In some embodiments lipid vesicles, such as liposomes, may be prepared from either a natural and/or synthetic phosphocholine-containing lipid having either two fatty acid chains of from about 12 to 20 carbon atoms, or one fatty acid chain of from about 12 to 20 carbon atoms and a second chain of at least about 8 carbon atoms. In some embodiments synthetic lipids are preferred as they may have fewer impurities. Suitable synthetic lipids include but are not limited to dimyristoylphosphatidylcholine, dioleoylphosphatidylcholine, dipalmitoylphosphatidylcholine and distearoylphosphatidylcholine. Suitable natural lipids include but are not limited to phosphatidylcholine and sphingomyelin. In some embodiments a liposome composition comprises a phosphatidylcholine, cholesterol and dihexadecyl phosphate although other liposome compositions will be apparent to the skilled artisan. Without being bound by theory, the liposomes can be biotinylated for stability purposes with, for example, biotin reagent (e.g., biotinoyl dipalmitoyl phosphatidylethanolamine (biotin-DPPE)). Compositions and methods for preparing liposomes are within the abilities of the skilled artisan. (see, e.g., U.S. Pat. Nos. 6,699,499, 6,696,079, 6,673,364, 6,663,885, 6,660,525, 6,623,671, 6,569,451, 6,544,958, 6,534,018 6,475,515, 6,468,798, 6,468,558, 6,465,008, 6,448,390, 6,436,435, 6,413,544, 6,387,614, 6,379,699, 6,372,720, 6,365,179, 6,358,752, 6,355,267, 6,350,466, 6,348,214, 6,344,335, 6,316,024, 6,290,987, 6,284,267, 6,271,206, 6,652,850, 6,660,525, 6,673,364, 6,696,079, 6,699,499, 6,706,861, 6,726,925, 6,733,777, 6,740,335, 6,743,430).

In some embodiments of the disclosed methods, a target analyte and/or an inhibitor thereof specifically binds to a binding partner. Therefore, in various exemplary embodiments a ligand/binding partner complex may comprise a target analyte/binding partner and/or a inhibitor/binding partner complex. Thus, “binding partner”, “binding ligand”, “ligand” and grammatical equivalents herein refer to a molecule or compound that interacts and specifically binds to at least one other molecule or compound. Therefore, the skilled artisan will appreciate that in some embodiments, one binding partner also may be a ligand and of another binding partner.

By “specifically bind” and grammatical equivalents herein are meant binding with specificity sufficient to differentiate at least one component under the binding conditions. In some embodiments, the binding can be sustained under the conditions of the assay, including but not limited to steps to remove or prevent non-specific binding and unbound ligand or binding partner. Non-limiting examples of ligand binding include but are not limited to antigen-antibody binding (including single-chain antibodies and antibody fragments, e.g., FAb, F(ab)′₂, Fab′, Fv, etc. (Fundamental Immunology 47-105 (William E. Paul ed., 5^{th} ed., Lippincott Williams & Wilkins 2003)), hormone-receptor binding, neurotransmitter-receptor binding, polymerase-promoter binding, substrate-enzyme binding, inhibitor-enzyme binding (e.g., sulforhodamine-valyl-alanyl-aspartyl-fluoromethylketone (SR-VAD-FMK-caspase(s) binding), allosteric effector-enzyme binding, biotin-streptavidin binding, digoxin-antidigoxin binding, carbohydrate-lectin binding, Annexin V-phosphatidylserine binding (Andree et al., 1990, J. Biol. Chem. 265(9):4923-8; van Heerde et al., 1995, Thromb. Haemost. 73(2):172-9; Tait et al., 1989, J. Biol. Chem. 264(14):7944-9), nucleic acid annealing or hybridization, or a molecule that donates or accepts a pair of electrons to form a coordinate covalent bond with the central metal atom of a coordination complex. In some embodiments the dissociation constant of the binding ligand can be less than about 10⁻⁴-10⁻⁶ M⁻¹, with less than about 10⁻⁵ to 10⁻⁹ M⁻¹ being preferred and less than about 10⁻⁷-10⁻⁹ M⁻¹ being particularly preferred. Determining the conditions to provide suitable binding is within the abilities of the skill artisan (see, e.g., Fundamental Immunology 69-105 (William E. Paul ed., 5^{th} ed., Lippincott Williams & Wilkins 2003).

In various embodiments, one or more of the reactants and/or products of the methods disclosed herein can be directly or indirectly conjugated to a moiety suitable for producing a detectable signal. Therefore, any one or more of a target analyte, an inhibitor, a binding partner, a detectable moiety, and the like may comprise or be conjugated to a detectable moiety. By “conjugated” and grammatical equivalents herein are meant bound to another molecule or compound. By “directly conjugated” and grammatical equivalents herein are meant bound without interposition of another molecule or compound. Thus, directly bound includes but is not limited to covalently bound, ionically bound, non-covalently bound (e.g., ligand binding as described above) without the interposition of another molecule or compound. “Indirectly conjugated” refers to two or more bound with the interposition of another molecule or compound. Thus, indirectly bound includes but is not limited to “sandwich” type assays, as known in the art.

By “detectable moiety”, “label”, “tag” and grammatical equivalents herein are molecules or compounds that are capable of being detected. Non-limiting examples of detectable moieties include isotopic labels (e.g., radioactive or heavy isotopes), magnetic labels (e.g. magnetic bead); physical labels (e.g., microparticle); electrical labels; thermal labels; colored labels (e.g., chromophores), luminescent labels (e.g., fluorescers, phosphorecers, chemiluminescers), quantum dots (e.g., redox groups, quantum bits, qubits, semiconductor nanoparticles, Qdot® particles (QuantumDot Corp., Hayward, Calif.)), enzymes (e.g., horseradish peroxidase, alkaline phosphatase, luciferase (Ichiki et al., 1993, J. Immunol. 150(12):5408-5417), β-galactosidase (Nolan et al., 1988, Proc Natl Acad Sci USA 85(8):2603-2607)), antibodies, and chemically modifiable moieties. Various examples of detection systems are described, for example, in Sambrook et al., Molecular Cloning: A Laboratory Manual A9.1-A9.49, 18.81-18.83 (3d. ed. Cold Spring Harbor Laboratory Press).

By “fluorescent moiety”, “fluorescent label”, and grammatical equivalents herein are meant a molecule that may be detected via its fluorescent properties. Suitable fluorescent labels include, but are not limited to, fluorescein, rhodamine, tetramethylrhodamine, tetramethyl rhodamine isothiocyanate (TRITC; Darzynkiewicz et al., 1992, Cytometry 13:795-808; Li et al., 1995. Cell Prolif. 238:571-9), eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, phycoerythrin, LC Red 705, Oregon green, Alexa-Fluors (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R- and B-phycoerythrin (PE), FITC, (Pierce, Rockford, Ill.), Cy 3, Cy5, Cy5.5, Cy7 (Amersham Life Science, Pittsburgh, Pa.) and tandem conjugates, such as but not limited to, Cy5PE, Cy5.5PE, Cy7PE, Cy5.5APC, Cy7APC. Suitable fluorescent labels also include, but are not limited to quantum dots. Suitable fluorescent labels also include self-fluorescent molecules, for example, green fluorescent protein (GFP; Chalfie et al., 1994, Science 263(5148):802-805; and EGFP; Clontech-Genbank Accession Number U55762), blue fluorescent protein (BFP; Quantum Biotechnologies, Inc., Montreal, Canada; Stauber, 1998, Biotechniques 24(3):462-471; Heim et al., 1996, Curr. Biol. 6:178-182), enhanced yellow fluorescent protein (EYFP; Clontech Laboratories, Inc., Palo Alto, Calif.), red fluorescent protein (DsRED; Clontech Laboratories, Inc., Palo Alto, Calif.), enhanced cyan fluorescent protein (ECFP; Clontech Laboratories, Inc., Palo Alto, Calif.), and renilla (WO 92/15673; WO 95/07463; WO 98/14605; WO 98/26277; WO 99/49019; U.S. Pat. Nos. 5,292,658; 5,418,155; 5,683,888; 5,741,668; 5,777,079; 5,804,387; 5,874,304; 5,876,995; 5,925,558). Further examples of fluorescent labels are found in Haugland, “Handbook of Fluorescent Probes and Research, Sixth Edition” (ISBN 0-9652240-0-7).

In some embodiments a fluorescent moiety may be an acceptor or donor molecule of a fluorescence energy transfer (FET) or fluorescent resonance energy transfer (FRET) system. As known in the art, these systems utilize distance-dependent interactions between the excited states of two molecules in which excitation energy can be transferred from a donor molecule to an acceptor molecule. (see Bustin, 2000, J. Mol. Endocrinol. 25:169-193; WO 2004/003510) Thus, these systems are suitable for methods in which changes in molecular proximity occur, such as, ligand binding as described above. Thus in some embodiments, a target analyte or inhibitor may comprise a donor and another a binding partner may comprises a suitable acceptor. Various permutations of the donor/acceptor arrangements will be apparent to the skilled artisan.

In some embodiments, the transfer of energy from donor to acceptor may result in the production of a detectable signal by the acceptor. In some embodiments, the transfer of energy from donor to acceptor may result in quenching of a fluorescent signal produced by the donor. Exemplary donor-acceptor pairs suitable for producing a fluorescent signal include but are not limited to fluorescein/tetramethylrhodamine, IAEDANS/fluorescein, EDANS/dabcyl, fluorescein/QSY 7, and fluorescein/QSY 9. Exemplary embodiments of donor-acceptor pairs suitable for quenching a fluorescent signal include but are not limited to FAM/DABCYL, HEX/DABCYL, TET/DABCYL, Cy3/DABCYL, Cy5/DABCYL, Cy5.5/DABCYL, rhodamine/DABCYL, TAMRA/DABCYL, JOE/DABCYL, Rox/DABCYL, Cascade Blue/DABCYL, Bodipy/DABCYL.

In some embodiments a detectable moiety can be a stain or dye. By “stain”, “dye” and grammatical equivalents herein refer to a substance or molecule that penetrates into or can be absorbed or taken up by another molecule or structure. In some embodiments, a strain or dye can be taken up by a specific class or type of compound or particle, e.g., nucleic acid (DNA or RNA), polypeptide, carbohydrate, a cell type and the like. Thus, in various exemplary embodiments, a stain can be a a vital stain (e.g. Trypan Blue, Neutral Red, Janus Green, Methylene Blue, Bismarck Brown, Cresyl Blue Brilliant, FM 4-64 (Pogliano et al. 1999, Mol Microbiol. 31(4): 1149-59) carboxyfluoroscein succinimidyl ester (CFSE), eosin Y, LDS-751 (U.S. Pat. No. 6,403,378), 7-amino-actinomycin D (AAD;), a nucleic acid stain (e.g., ethidium bromide, LDS 751, GelStar® nucleic acid stain (Cambrex Corp., East Rutherford, N.J.), SYBR® Green I and II (Molecular Probes, Inc., Eugene, Oreg.), SYTO blue, green, orange and red (Molecular Probes, Inc., Eugene, Oreg.), SYTOX® blue, green and orange (Molecular Probes, Inc., Eugene, Oreg.), propidium iodine (Molecular Probes, Inc., Eugene, Oreg.), Vistra Green™ (GE Healthcare Technologies, Waukesha, Wis.)), and/or a protein stain (Deep Purple™ (GE Healthcare Technologies, Waukesha, Wis.), SYPRO ruby, red, tangerine and orange (Molecular Probes, Inc., Eugene, Oreg.), Coomassie fluor orange (Molecular Probes, Inc., Eugene, Oreg.) and combinations thereof (e.g., ViaCount® (Guava Technologies, Hayward, Calif.) Guava Technologies Inc. Technical Note. Guava ViaCount®. Doc. part no. 4600-0520). Non-limiting examples of cell viability assay reagents are described in WO02/088669. Further examples of stains and dyes are found in Haugland, “Handbook of Fluorescent Probes and Research, Sixth Edition” (ISBN 0-9652240-0-7).

In some embodiments a target analyte may synthesize or produce a compound capable of producing a detectable signal. For example, in embodiments in which a target analyte or inhibitor can be a cell or is cell-associated, the cell may express a compound capable of producing a detectable signal. As the skilled artisan is aware, a compound capable of producing a detectable signal can be expressed either alone or in combination with other compounds (e.g., as a fusion polypeptide), and expression may be inducible or constitutive, as known in the art. Non-limiting examples of compounds suitable for such expression include but are not limited to horseradish peroxidase, alkaline phosphatase, luciferase, β-galactosidase, BFP, DsRED, ECFP, EGFP; GFP; EYFP, and renilla, as described above. In some embodiments polypeptides capable of producing a detectable signal may be introduced into the cells as siRNA, a plasmid, nucleic acids, or polypeptides.

The target analytes may be obtained from any source. For example, a target analyte may be isolated or enriched from a sample, or be analyzed in a raw sample. Thus, a sample includes but is not limited to, a cell, a tissue (e.g., a biopsy), a biological fluid (e.g., blood, plasma, serum, cerebrospinal fluid, amniotic fluid, synovial fluid, urine, lymph, saliva, anal and vaginal secretions, perspiration, semen, lacrimal secretions of virtually any organism, with mammalian samples being preferred and human samples being particularly preferred), an environment (e.g., air, agricultural, water, and soil samples)), research samples (e.g., tissue culture sample, a bead suspension, a bioreactor sample). In addition to the target analyte, in some embodiments the sample may comprise any number of other substances or compounds, as known in the art. In some embodiments, sample refers to the original sample modified prior to analysis by any steps or actions required. Such preparative steps may include washing, fixing, staining, diluting, concentrating, decontaminating or other actions to facilitate analysis.

Once a sample is obtained, it can be analyzed by the disclosed methods. Therefore, in some embodiments the presence or absence of one or more target analytes can be determined, the quantity of one or more target analytes can be determined, and/or a characteristic of a target analyte can be determined (e.g, the binding affinity of a target analyte and a binding partner).

In some embodiments, a sample can be analyzed under competitive binding conditions, as described above. In some embodiments, competitive binding conditions can be established by reacting a sample that may contain one or more target analytes with one or more binding partners followed by the addition of one or more inhibitors. In some embodiments, competitive binding conditions can be established by reacting the inhibitor(s) with the binding ligand(s) followed by the addition of the sample(s). In some embodiments, the sample(s) and inhibitor(s) can react simultaneously with the binding ligand(s). In some embodiments, each binding ligand can be labeled with one or more detectable moieties. In some embodiments, the signal produced by each detectable moiety can be distinguished. Determining the reaction conditions for the addition of the various components is within the abilities of the skilled artisan. However, generally, each reaction step can occur at or about room temperature for about 20 to about 30 minutes. The temperature, pH, isotonicity, reaction period and other conditions can depend at least in part upon the sample, the composition of the target analyte(s), inhibitor(s), and binding ligand(s). Determining such conditions is within the abilities of the skilled artisan.

To analyze the extent of inhibition, the amount of target analyte and/or inhibitor bound by the binding partner can be determined. In some embodiments, the extent of inhibition can be compared to control experiments in which known amounts of binding partner, inhibitor, and target analyte react under competitive binding conditions. In some embodiments, the extent of inhibition can be determined by comparing the results obtained with a sample to a calibration curve obtained by reacting known amounts or titrating known amounts of binding partner, inhibitor, and/or target analyte under competitive binding conditions. In some embodiments, the binding partner can be directly or indirectly conjugated to a detectable moiety. For example, in embodiments wherein the binding partner can be an antibody, the antibody can be indirectly conjugated to a detectable moiety by being bound by an anti-antibody comprising a detectable moiety. In embodiments, wherein the inhibitor comprises a microparticle, the antibody bound to the inhibitor also can be construed to be labeled with the microparticle. Thus, a binding partner can be directly and/or indirectly labeled with various types of detectable moieties selected at the discretion of the practitioner. Selecting the number and types of detectable moieties is within the abilities of the skilled artisan.

In some embodiments, at least first and second target analytes can be analyzed. In some embodiments, a first target analyte may be a cell or a cell-associated analyte (ca-target analyte) and a second target analyte may not be cell-associated (na-target analyte). In some embodiments, such first and second target analytes can be analyzed in a single reaction vessel. For example, a first target analyte can be a component of a cell in a culture and a second target analyte can be found in the culture media. Therefore, in some embodiments a first target analyte can be a receptor, a marker, antigen on a cell membrane (e.g., a T-cell, B-cell, neutrophil, hybridoma), or can be on the cell interior. Therefore, in some embodiments a binding partner can comprise moieties for the delivery and internalization of the binding partner into a cell. For example in some embodiments a binding partner can be delivered to a cell within a liposome (e.g., Lipofectamine™ 2000, PLUS™ Reagent, Lipofectamine™, DMRIE-C, Cellfectin®, Lipofectin®, Oligofectamine™ (Invitrogen, Carlsbad, Calif.)), which in some embodiments, can comprise cell targeting moieties. (e.g., U.S. Pat. Nos. 6,339,070, 6,780,856, 6,693,083, 6,645,490, 6,627,197, 6,599,737, 6,565,827, 6,500,431, 6,287,537, 6,251,866, 6,232,295, 6,168,932, 6,090,365, 6,015,542, 6,008,190, 5,994,317, 5,843,398, 5,595,721) In some embodiments, a cell (e.g., phagocytic cell (e.g., macrophage)) may internalize a binding partner without the use of a cell targeting moiety. In some embodiments, the binding partner to be internalized may comprise a microparticle. In some embodiments, a second target analyte can be an antibody (e.g., a monoclonal antibody), cytokine (e.g., IL-1 to −15), or other molecule or compound secreted by a cell (e.g., a hormone). In some embodiments, a ca-target analyte can be a precursor or cell-associated form of the na-target analyte. To analyze the target analytes, they can be bound to first and second binding partners, respectively. In various exemplary embodiments, the specificity of the binding partners can be substantially unique or can be substantially equivalent. The binding partners can be directly or indirectly conjugated to one or more detectable moieties. For example, in some embodiments a first binding ligand may comprise a fluorescent moiety, a second binding ligand may comprise fluorescent moiety and a microparticle, and a cell can be labeled with a dye or stain.

In some embodiments, the activity of a target analyte can analyzed. Therefore, in some embodiments, a microparticle may comprise a substrate or an inhibitor of the activity of a target analyte and may be modified in the presence of the target analyte. The modification of the substrate and/or inhibitor may result in a change in the production of a detectable signal. Therefore, in some embodiments, a change in a detectable signal may be an increase or decrease in detectable signal. For example, in some embodiments a substrate attached to a microparticle may be fluorescently labeled and the action of the target analyte may release the fluorescent label from the substrate resulting in a decrease in fluorescence associated with the microparticle. In some embodiments, the substrate can be a protease (e.g., a metalloprotease) released by a cell and the substrate can be a fluorescently labeled peptide. Hydrolysis of the peptide by the protease may result in decreased fluorescence associated with the microparticle. In some embodiments, the target analyte can be kinase or a phosphatase and the addition and/or removal of a phosphate group from the microparticle bead can result in an increase or decrease in detectable signal. The skilled artisan can appreciate that the use of moieties that produce distinguishable detectable signals can be used to analyze multiple target analytes in a single reaction vessel.

Once the products of the various methods are made (e.g., target analyte/binding partner complex, inhibitor/binding partner complex, stained cell, etc.) and comprise one or more detectable moieties, they can be analyzed by various methods as known in the art. In some embodiments, analysis can be visual inspection (e.g., light microscopy) and/or automated detection and/or quantitation and/or sorting. For example, in some embodiments analysis can employ a automated detection system in which a signal produced by a detectable moiety can be optically linked to the detection system. Such systems are known in the art and include but are not limited to systems capable of analyzing light scatter, radioactivity, and/or luminescence (e.g., fluorescence, phosphorescence, chemiluminescence). In various exemplary embodiments, the products of the methods disclosed herein can be analyzed as a population and/or can be individually analyzed. For example, in some embodiments, the products disclosed herein can be analyzed by flow cytometry (see e.g., U.S. Pat. Nos. 4,500,641, 4,665,020, 4,702,598, 4,857,451, 4,918,004, 5,073,497, 5,089,416, 5,092,989, 5,093,234, 5,135,302, 5,155,543, 5,270,548, 5,314,824, 5,367,474, 5,395,588, 5,444,527, 5,451,525, 5,475,487, 5,521,699, 5,552,885, 5,602,039, 5,602,349, 5,643,796, 5,644,388, 5,684,575, 5,726,364, 5,726,751, 5,739,902, 5,824,269, 5,837,547, 5,888,823, 6,079,836, 6,133,044, 6,263,745, 6,281,018, 6,320,656, 6,372,506, 6,411,904, 6,542,833, 6,587,203, 6,594,009, 6,618,143, 6,658,357, 6,713,019, 6,743,190, 6,746,873, 6,780,377, and 6,782,768), scanning cytometry (see, e.g., U.S. Pat. No. 6,275,777), and/or microcapillary cytometry (see e.g., U.S. patent application Ser. No. 09/844,080, and U.S. Provisional Patent Application Ser. No. 60/230,380; and the Guava PCA, Guava Technologies, Hayward, Calif.), incorporated by reference.

In the present application, use of the singular includes the plural unless specifically stated otherwise. All literature and similar materials cited in this application, including but not limited to patents, patent applications, articles, books, and treatises regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls. Aspects of the present disclosure may be further understood in light of the following examples, which should not be construed as limiting the scope of the present disclosure in any way.

### 6. EXAMPLES

### Example 1

### Insulin detection by a Competitive Bead Based Assay

Microsphere polystyrene beads (carboxyl 4-6 μm) (Catalog No. 234, 237 Bangs Laboratories, Fishers, Ind.; Spherotech, Inc., Libertyville, Ill.) were covalently coated with purified recombinant human insulin (rhI, Catalog No. I2767, Sigma-Aldrich, St. Louis, Mo.) (see, Kono, 1988, Vitam. Horm. 7:103-154; Morihara, et al., 1979, Nature 280:412-413; Smith, 1996, Am. J. Med. 40:662-666) via EDC/DADPA (Prod. No. 53154 Doc. No. 0522, Prod. No. 44899 Doc No. 0480, Pierce Biotechnology, Inc., Rockford, Ill.) using the method recommended by the manufacturers. (see Ajuh, et al., 2000, EMBO 19:6569-6581; Giles, et al., 1990, Anal. Biochem. 184:244-24; Grabarek, et al., 1990, Anal. Biochem. 185:244-28; Lewis, et al., 2000, Endocrinology 141:3710-6; Williams, et al., 1981, J. Am. Chem. Soc. 103:7090-7095; Yoo, et al., 2002, J. Biol. Chem. 277:15325-32) Excess, rhI was used to saturate available attachment sites.

For the competitive binding assay, various amounts of rhI (0 U/mL, 500 μU/mL, 1 mU/mL, 10 mU/mL, 50 U/mL, 100 mU/mL) were incubated with mouse anti-human insulin MAb (1′Ab, 20 μl/test, mouse IgG) (BD Biosciences, Franklin Lakes, N.J.)) for 30 min. at room temperature in 1×PBS with BSA and azide (PBS-BA). Microparticle beads containing rhI were added and the reaction mixture was incubated for 30 min. at room temperature. Goat anti-mouse PE-labeled antibody (2′Ab) (Catalog No. 4700-0010, Guava Technologies, Inc., Hayward, Calif.) was added and the solution was incubated at for 30 min. at room temperature.

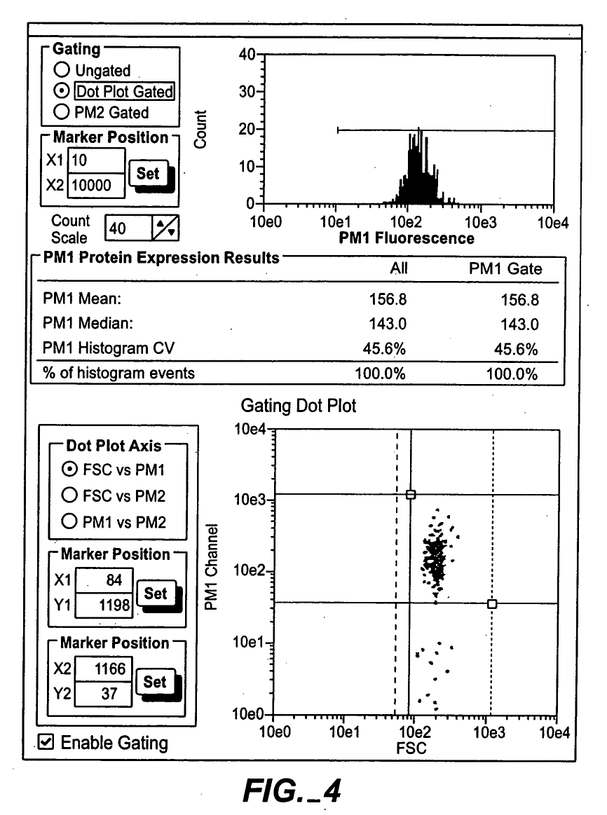

The beads were washed to remove unbound 1′Ab and 2′Ab antibodies by centrifugation for 8 min. at 1300 rpm in 1×PBS. The pelleted microparticle beads were resuspended in 1×PBS and analyzed using a Guava PCA microcapillary cytometer (Guava Technologies, Inc., Hayward, Calif.). Instruments settings used according to manufacturer's recommendations as the protocol for express reagents, where the gain for PM1 by first running negative samples and negative controls to insure reading of less than 10 MFI (mean fluorescence intensity). This is followed by test samples (see FIG. 4) and adjusting the PM1, usually around 410. This varies from instrument to instrument depending on the age of the laser excitation source. For each assay, fluorescence was recorded as mean and median MFI. An isotype matched control at 10× the concentration of test antibody was run in parallel as the 1′Ab. A negative control also was run in parallel and did not utilize a 1′Ab.

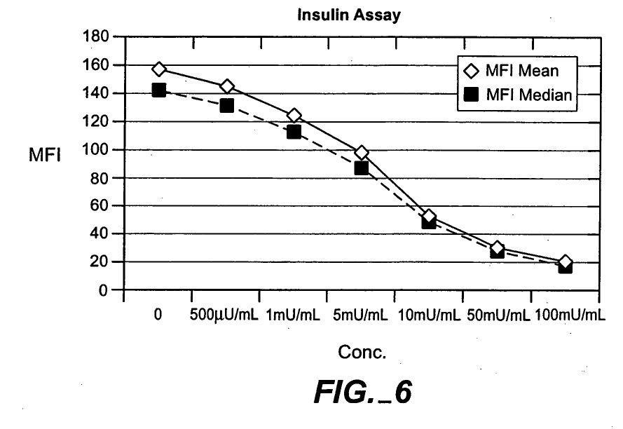

As shown in FIG. 6, a graph of MFI vs. increasing concentration of free rhI resulted in decreased fluorescence. Therefore, the free rhI and rhI coated microparticles competed for binding with the 1′Ab. As a result, less 1′Ab and 2′Ab bound in a sandwich fashion to the rhI coated beads and less fluorescence was detected.

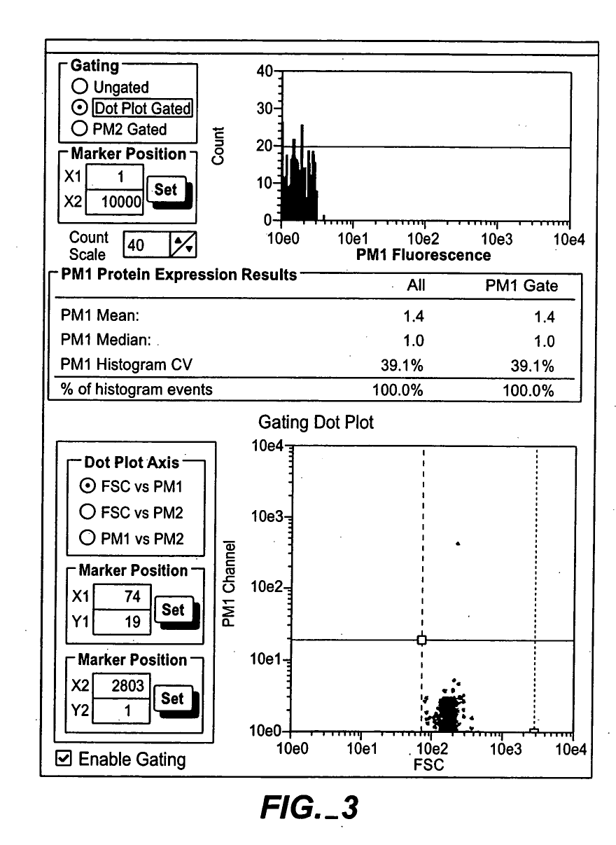

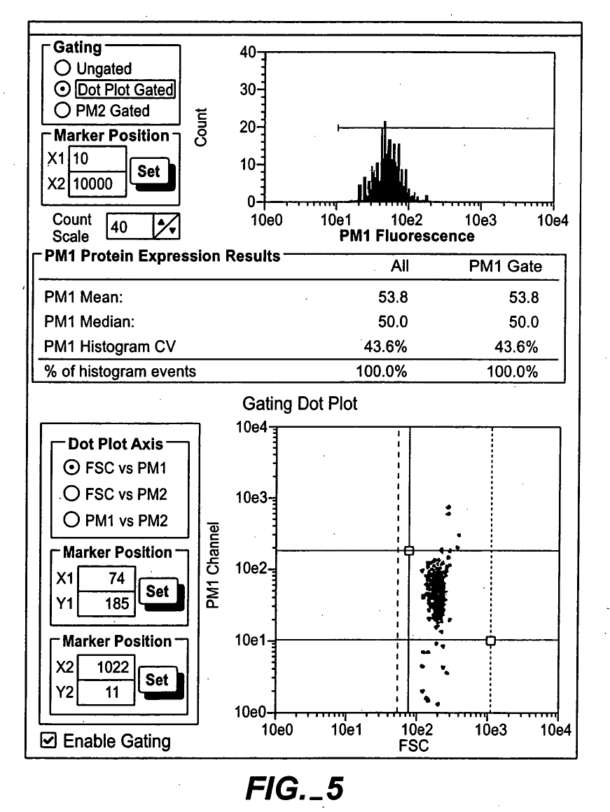

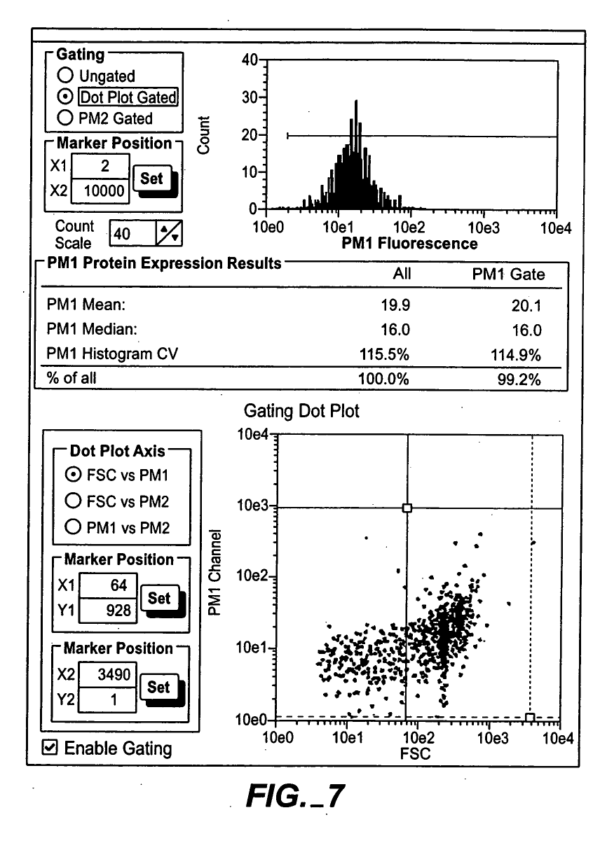

FIGS. 2 and 3 show the results of the isotype and negative controls, respectively. The beads detected in these figures are easily distinguished from the competitive binding assay in which no free rhI was available for 1′Ab binding (FIG. 4). However, as the amount of free rhI is increased to 10 μU/mL (FIG. 5), the detected beads shifts down due to the decreased fluorescence signal. Doublets were advantageous not detected (see, FIG. 7).

### Example 2

### Antibody Screening

A competitive binding assay is done using various amounts of rhI (0 U/mL, 500 μU/mL, 1 mU/mL, 10 mU/mL, 50 U/mL, 100 mU/mL) and mouse anti-human insulin MAb (1′Ab) as described in Example 1. To determine if an unknown antibody binds to insulin, a competitive binding assay is performed using an equivalent amount of an unknown antibody as 1′Ab. By graphing the results and comparing the curves obtained with the anti-human insulin and the unknown antibody, relative affinity of the unknown antibody is determined.

To screen an unknown antibody for insulin binding, a unknown human antibody is titrated and incubated with insulin-coated microparticles for about 30 min. at room temperature. The microparticles are centrifuged, washed, and resuspended as described above. The 1′Ab (mouse anti-insulin IgG) is added and the mixture is incubated, washed, and resuspended as described above. A 2′Ab (PE labeled goat anti-mouse) is added and the mixture is incubated, washed, and resuspended as described above. The labeled complexes are analyzed by a Guava PCA micocapillary cytometer. A decrease in signal compared to negative controls is indicative that the unknown antibody binds to insulin and inhibits 1′Ab binding.

### Example 3

### Analysis of Multiple Target Analytes by Competitive Bead Based Assay

Two antigens, rhI and recombinant human erythropoietin (rhEPO: Catalog No. E5627, Sigma-Aldrich, Inc., St. Louis, Mo.) (see Bailey, et al., 1991, J. Biol. Chem. 266:24121; Davis, et al., Biochemistry 26:2633; Dordal, et al., 1985, Endocrinology 116:2293; Hanspal, et al., 1991, J. Biol. Chem. 266:15626; Miyake, et al., 1977, J. Biol. Chem. 252:5558), are each bound to microsphere polystyrene beads having different diameters, 6 μm and 11 μm, respectively via EDC/DADPA (two step procedure).

For the competitive binding assay, various amounts of rhI and rhEPO are incubated with a mouse anti-human insulin MAb (1′Abᵢ) and a goat anti-human EPO MAb (1′Abₑ) (IgG₁/k, Catalog No. 01300, STEMCELL Technologies, Inc., Vacouver, BC; see Wognum, et al., 1988, Blood 71:1731-1737 for 30 min. at room temperature in 1×PBS. Microparticle beads containing either with rhI or rhEPO are added and the reaction mixture is incubated for 30 min. at room temperature. Rabbit anti-mouse PE-labeled antibody and Rabbit anti-goat FITC labeled antibody (2′Abs) are added and the solution is incubated at for 30 min. at room temperature.

The microparticle beads are washed to remove unbound 1′Abᵢ, 1′Ab_{E} and 2′Abs by centrifugation for 8 min. at 1300 rpm. The pelleted microparticle beads are resuspended in 1×PBS and are analyzed using a Guava PCA microcapillary cytometer (Guava Technologies, Inc., Hayward, Calif.). For each assay, forward light scattering and FITC and PE fluorescence is recorded. The results indicate that multiplex competitive binding assays can be performed by the disclosed methods. Isotype matched controls are run in parallel for 1′Abᵢ and 1′Ab_{E}. A negative control also is run in parallel that did not utilize a 1′Ab.

Human TNF-α and IFN-γ are analyzed in the above protocol using microparticles containing TNF-αor IFN-γ, mouse anti-human TNF-α antibody (Catalog No. 4T10, HyTest Ltd., Turku, Finland) and mouse anti-human IFN-γ antibody (Catalog No. 4I22, HyTest Ltd., Turku Finland). Because the 1′Abs are both mouse, the complexes formed by 1′Ab binding are discriminated by the microparticles containing TNF-α or IFN-γ being distinguishable by each having a distinguishable fluorescent dye contained therein or by the microparticles having a diameter that is distinguishable by a microcapillary cytometer (Guava PCA).

### Example 4

### Viral Load Determination

gp120 is a glycoprotein of human immunodeficiency virus (HIV) that is exterior to the viral lipoprotein envelope. Therefore, gp120 can be used in a competitive bead based assay to detect HIV virions in biological samples. gp120 from HIV-1 (Catalog No. 2003LAV, Protein Sciences Corp., Meriden, Conn.) is coupled to microsphere polystyrene beads using the via EDC/DADPA (two step procedure). For the competitive binding assay, a sample of a biological fluid is serially diluted half-log from 10^{-0.5} to 10⁻⁶ in 1×PBS-BA. A mouse anti-gp120 MAb (Catalog No. MMS-193P, Covance Research Products, Berkeley, Calif.) is added to each dilution and incubated for 30 min. at room temperature. Microparticle beads coated with gp120 are added and the reaction mixture is incubated for 30 min. at room temperature. Goat anti-mouse PE-labeled antibody (2′Ab) is added and the solution is incubated for 30 min. at room temperture.

The beads are washed to remove unbound 1′Ab and 2′Ab antibodies by centrifugation for 8 min. at 1300 rpm. The pelleted beads are resuspended in 1×PBS and are analyzed using a Guava PCA microcapillary cytometer (Guava Technologies, Inc., Hayward, Calif.). For each assay, fluorescence is recorded as mean and median MFI. An isotype control is run in parallel using an isotype matched mouse anti-insuling antibody as the 1′Ab. A negative control also is run in parallel and did not utilize a 1′Ab. A change in fluorescence intensity that is inversely proportional to the dilution of the biological sample is indicative of HIV-1 gp120 being present in the biological sample.

### Example 5

### Simultaneous Analysis of Cells and Beads

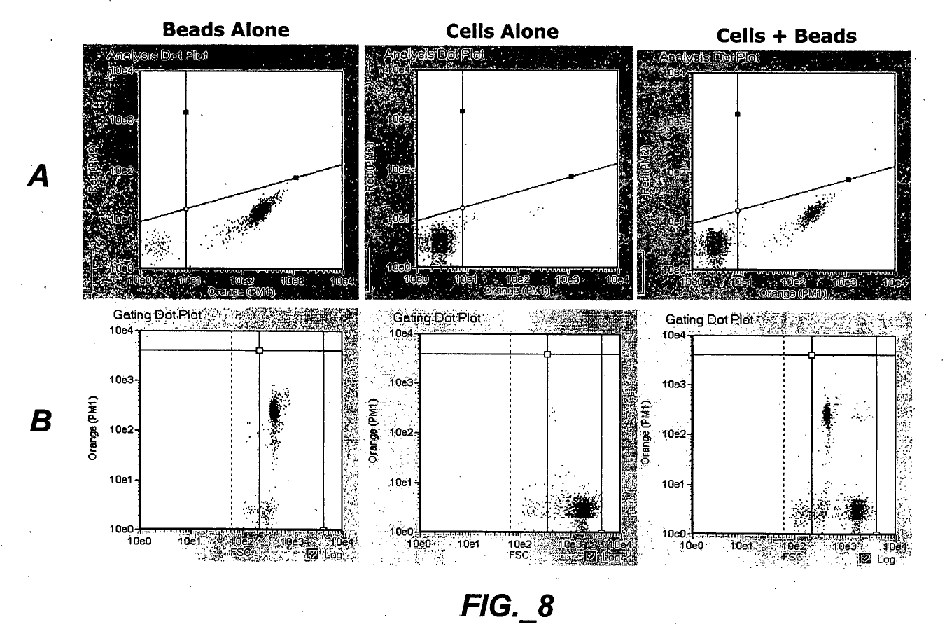

Cells were normal Jurkat cells with no fluorescent label or stain. Beads were obtained from Bangs Labs (Quantum MESF PE beads, Catalog 827A, Fishers, Ind.). Cells and beads were pipetted together and analyzed on a microcapillary cytometer (Guava PCA-96, Hayward, Calif.). Beads and cells were distinguished based on light scatter using a microcapillary cytometer (Guava PCA) (FIG. 8B).

Normal Jurkat cells were stained with propidium iodide (PI) (see, e.g, Caballero et al., 2004, Reprod. Domest. Anim. 39(5):370-375; Armeni et al., 2004, Toxicology. 2004 Oct. 15; 203(1-3): 165-78) mixed with the fluorescent Quantum MESF PE beads and analyzed by a microcapillary cytometer (Guava PCA). Beads and cells were distinguished based on fluorescence (FIG. 8A).

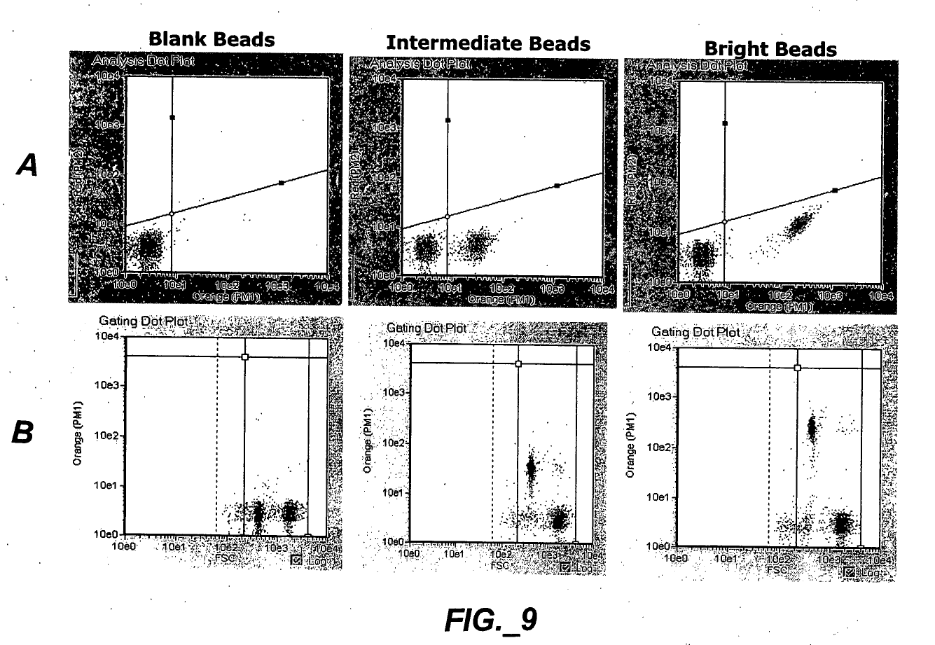

Normal Jurkat cells were simultaneously analyzed with beads with different amounts of PE conjugated to the bead surface (blank beads (non-fluorescent), intermediate fluorescence, bright fluorescence). As shown in FIGS. 9A (fluorescence) and 9B (light scatter), the data demonstrate that microcapillary cytometry (Guava PCA) distinguished and separated beads of various fluorescence intensities from cells.

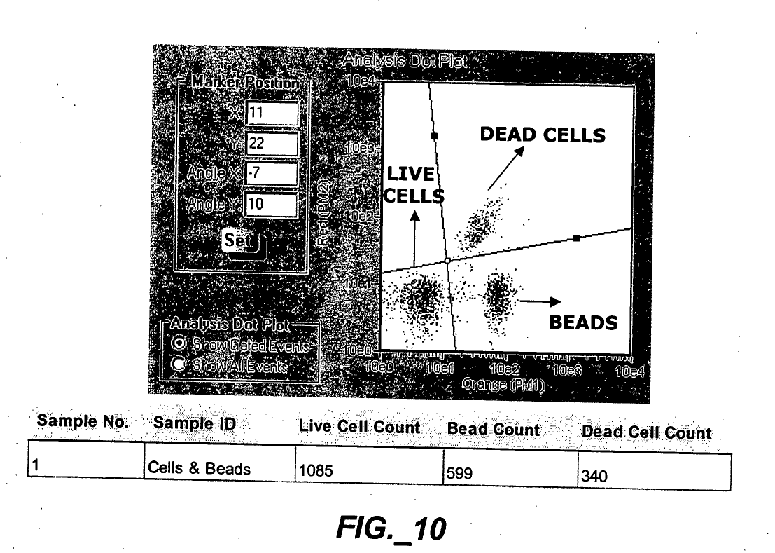

Normal Jurkat cells were combined with beads pre-labeled with a known quantity of PE for fluorescence detection and analytical separation. In addition, cells and beads were incubated with a fluorescent indicator of cell death, 7-actinomycin D (7-AAD). As shown in FIG. 10, live cells were separated from fluorescent beads along the horizontal axis, and dead cells were separated from both the live cells and labeled beads by microcapillary cytometry (Guava PCA). In the example shown, data was collected on 2000 events, as entered by the user in the Guava software.

### Example 6

### Simultaneous Analysis of Islet if Langerhan Cell Viability and Insulin Production

Pancreatic cells suitable for transplantation are obtained from a donor using aseptic surgical techniques. The insulin-producing islets of Langerhans cells are separated from the other cells in the pancreas using a Ricordi Chamber (Barshes et al., 2004, Transplant Proc. 36(4):1127-9; Goss et al., 2002, Transplantation. 74(12):1761-6) or other method (Field et al., 1996, Transplantation 61:1554; Linetsky et al., 1997, Diabetes 46:1120; U.S. Pat. Nos. 4,868,121, 5,273,904, 5,322,790, 5,447,863, 5,821,121) and cultured for 5-11 days (Rosenbaum et al., 1998, Proc. Natl. Acad. Sci. USA 95(13):7784-7788); U.S. Pat. No. 6,365,385).

To analyze the islet cells for viability and the supernatant for insulin production, a first species anti-donor insulin antibody labeled with a microparticle (made as described above) is added to a culture aliquot containing supernatant and islet cells. Following a 15-30 min. incubation at room temperature, beads and cells are gently centrifuged, washed, in media, resuspended in media and a second species anti-donor insulin PE labeled antibody and FITC labeled Annexin V (BD Biosciences, Franklin Lakes, N.J.) are added. Annexin V is a calcium dependent binding protein or binding partner of phospholipid phosphatidylserine (PS). During apoptosis PS is translocated from the inner to the outer portion of the plasma membrane, where it is able to bind Annexin V in the presence of Ca²⁺. (Vermes et al., 1995, J. Immunol. Meth. 184:39-51) Following a 15-30 min. incubation at room temperature, beads and cells are analyzed using a microcapillary cytometer (Guava PCA, Hayward, Calif.). By comparing the results to standards or testing a standard in parallel the quantity of insulin can be determined. The results also provide the number of viable, apoptotic cells, non-viable cells.

Rather than or in addition to Annexin V FITC labeling, islet cells are stained with propidium iodide (PI, BD Biosciences, Franklin Lakes, N.J.). PI binds dsDNA but crosses the plasma membrane of non-viable cells, which occurs late in apoptosis. PS translocation and Annexin V binding occurs early in apoptosis. Therefore, differential detection of PI and Annexin V is used to stage apoptosis. (Raynal et al., 1994, Biochim Biophys Acta. 1197(1):63-93; Martin et al., 1995, J Exp Med. 182(5):1545-56; Vermes et al., 1995, J. Immunol. Meth. 184:39-51).

### Example 7

### Simultaneous Analysis of Secreted Monoclonal Antibodies and Hybridoma Cells

A fixed number of goat anti-human labeled microbeads (20,000, Quantum anti-human antibody beads, Bangs Labs) are added to each well of a 96-well plate containing hybridoma cells. The plate is incubated at room temperature with agitation for 1 hr and centrifuged to 5000 rpm for 5 min. Supernatant is removed and cells and beads are resuspended. PE-labeled donkey anti-human antibody (Jackson Labs) is added to a final concentration of (5 ng/μl). Plates are incubated for 45 min at room temperature, beads and cells are pelleted, resuspended in 1×PBS, and analyzed by microcapillary cytometry. Hybridoma cell viability also can be determined using Annexin V and/or PI as described above and/or using LDS 751 (see, e.g., WO02/088669). The results are indicative of the amount of monoclonal antibody in the supernatant, the number of hybridoma cells producing monoclonal antibody, and the viability of the hybridoma cells. By using unlabeled and labeled antibodies specific for heavy or light chains (e.g., unlabeled antibody to K chains, labeled antibody to γheavy chains) the monoclonal antibodies are isotyped and clonal homogeneity is assessed. The secreted monoclonal antibodies and hybridoma cells are analyzed in further detail using labeled and/or unlabeled antibodies that are allotype and/or idiotype and/or xenotype specific.

Monoclonal antibody in hybridoma supernatants can be quantitated by establishing a calibration curve. Therefore, the results obtained with an unknown is compared to the calibration curve to quantify monoclonal antibody.

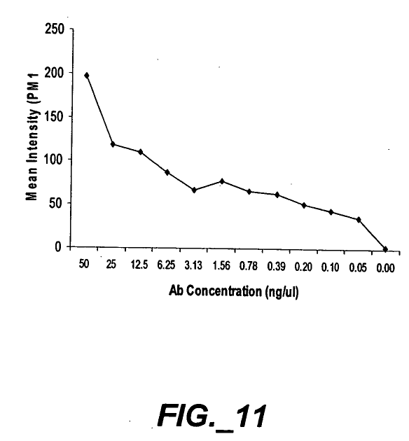

To establish a calibration curve, various known concentrations of human IgG (Jackson Labs) were added to individual wells of a 96-well plates. Goat anti-human labeled beads (20,000) were added to each well for a total volume of 100 μl. Plates were incubated at room temperature with agitation for 1 hr. The plate was centrifuged at 5000 rpm for 5 min to pellet the beads. The supernatant was removed and 100 μl of secondary PE labeled donkey anti-human IgG (5 ng/μl) was added to each well. Plates were incubated for 45 min at room temperature with agitation. Beads were pelleted, supernatant removed, and resuspended in 100 μl 1×PBS, and analyzed by microcapillary cytometry (Guava PCA). In the graph shown in FIG. 11, bead fluorescence was detected and was proportional to the concentration of human IgG. on the right, this concept was validated using antibodies pre-conjugated to fluorescent molecules for detection on the Guava platform, but the same information can be obtained using a secondary detection approach. As indicated on the plot above, the Guava platform is able to determine bead fluorescence, and that fluorescence decreases with decreasing amounts of antibody in solution.

1. A method of detecting target analytes comprising:
a) labeling, in a vessel, first and second target analytes with moieties capable of producing detectable signals, wherein said first analyte is cell-associated and said second target analyte is not cell-associated, and
b) detecting the signals produced by the labeled target analytes.

2. The method according to claim 1, wherein said first analyte is a precursor of said second analyte.

3. The method according to claim 1, wherein said first and second analytes independently comprise a peptide, a nucleic acid, a carbohydrate, a lipid, or combinations thereof.

4. The method according to claim 4, wherein said peptide is a monoclonal antibody.

5. The method according to claim 4, wherein said first and second target analytes are virus peptides, nucleic acids, or combinations thereof.

6. The method according to claim 6, wherein said virus is HIV.

7. The method according to claim 1, wherein said moieties are fluorescent moieties.

8. The method according to claim 1, wherein said labeled target analytes are capable of being distinguished.

9. The method according to claim 1, wherein said second target analyte is labeled by binding to a microparticle comprising at least one of said moieties.

10. The method according to claim 1, wherein said signals are fluorescent signals and are detected by a microcapillary cytometer optically linked to a fluorescence system.

11. A method of analyzing first and second target analytes, wherein said first analyte is cell-associated and labeled with a first moiety capable of producing a first fluorescent signal and said second analyte is not cell associated, and is labeled with a microparticle and a second moiety capable of producing a second fluorescent signal, comprising detecting the fluorescence signals produced as the labeled target analytes are drawn through a microcapillary cytometer that is optically linked to a fluorescence system.

12. The method according to claim 12, wherein said first analyte is a precursor of said second analyte.

13. The method according to claim 12, wherein said first and second analytes are monoclonal antibodies.

14. The method according to claim 12, wherein said fluorescence signals produced by said labeled analytes are capable of being distinguishable.

15. The method according to claim 12, wherein said labeled analytes are capable of being distinguished by said microcapillary cytometer.

16. A method of detecting a target analyte, comprising:
a) inhibiting antibody—target analyte binding with a microparticle comprising a competitive inhibitor of said target analyte, and
b) measuring the antibody bound to said competitive inhibitor as said microparticle is drawn through a microcapillary cytometer that is optically linked to a fluorescence system, whereby said target analyte is detected.

17. The method according to claim 17, wherein said antibody comprises a fluorescent moiety.

18. The method according to claim 17, further comprising labeling said antibody bound to said competitive inhibitor with a fluorescent moiety.

19. The method according to claim 19, wherein said antibody is labeled by binding said antibody to a second antibody comprising said fluorescent moiety.

20. The method according to claim 1, further comprising quantitating said target analyte.

21. The method according to claim 1, wherein said target analyte comprises a peptide, nucleic acid, carbohydrate, lipid, or combinations thereof.

22. The method according to claim 22, wherein said peptide is insulin.

23. The method according to claim 22, wherein said target analyte is HIV.

24. A method of detecting a target analyte, comprising:
a) reacting an antibody with a target analyte and a competitive inhibitor thereof under competitive binding conditions,
b) measuring the antibody bound to said competitive inhibitor as it is drawn through a microcapillary cytometer that is optically linked to a detection system.

25. The method according to claim 25, wherein said antibody is labeled with a fluorescent moiety.

26. The method according to claim 25, wherein said antibody bound to said competitive inhibitor is labeled by binding to a second antibody comprising a fluorescent moiety.

27. The method according to claim 25, wherein said competitive inhibitor comprises a microparticle.

28. The method according to claim 26, wherein said target analyte comprises a peptide, nucleic acid, carbohydrate, lipid, or combinations thereof.

29. The method according to claim 28, wherein said peptide is insulin.

30. The method according to claim 28, wherein said target analyte is HIV.
